(19) United States
(12) Patent Application Publication
(10) Pub. No.: US 2006/0094701 A1
(43) Pub. Date: May 4, 2006

(54) Use of beta-2 bronchodilator drugs

(76) Inventor: John Morley, Muttenz (CH)

Correspondence Address: HESLIN ROTHENBERG FARLEY & MESITI PC, 5 COLUMBIA CIRCLE, ALBANY, NY, 12203, US

(21) Appl. No.: 11/303,277

(22) Filed: Dec. 16, 2005

Related U.S. Application Data
(63) Continuation of application No. 10/821,553, filed on Apr. 9, 2004, now abandoned.
Application No. 10/821,553 is a continuation of application No. 10/095,846, filed on Mar. 12, 2002, now abandoned.
Application No. 10/095,846 is a continuation of application No. 09/550,039, filed on Apr. 14, 2000, now abandoned.
Application No. 09/550,039 is a continuation of application No. 08/972,526, filed on Nov. 18, 1997, now abandoned.
Application No. 08/972,526 is a division of application No. 08/382,744, filed on Feb. 2, 1995, now abandoned.
Application No. 08/382,744 is a continuation of application No. 08/223,798, filed on Apr. 6, 1994, now abandoned.
Application No. 08/223,798 is a continuation of application No. 07/862,907, filed on Apr. 3, 1992, now abandoned.

(30) Foreign Application Priority Data
Apr. 5, 1991 (GB) 9107196.9

(51) Int. Cl. A61K 31/33; A61K 31/138

(52) U.S. Cl. 514/183; 514/651

(57) ABSTRACT

Improved use of selective β₂ sympathomimetic bronchodilator drugs in the therapy of obstructive or inflammatory airways disease, e.g. asthma, comprises use in enantiomeric rather than conventional racemic form. The improved use reduces occurrence of side effect, e.g. exacerbation of basal disease status or compromise or deterioration of lung function.

The present invention relates to a new and improved use of selective β₂ sympathomimetic bronchodilator drugs in the therapy of obstructive or inflammatory airways disease, especially asthma.

Bronchodilator drugs employed in the therapy of obstructive or inflammatory airways disease, e.g. asthma, are divisible into three classes:
1. Adrenergic or sympathomimetic drugs (the terms “adrenergic” and “sympathomimetic” are used in the art interchangeably);
2. Anticholinergic drugs; and
3. Methylxanthine drugs.

The present invention is concerned with the first of these drug classes.

The adrenergic or sympathomimetic drugs are so called because they are understood to exert their effect through their action on the body's adrenergic receptors of which there are three functionally divided types, the α, β₁ and β₂ receptors. On the basis of their interaction with these three receptor types, the adrenergic or sympathomimetic drugs are in turn classifiable into three groups:
1.1 Non-selective sympathomimetic drugs;
1.2 Non-selective β sympathomimetic drugs; and
1.3 Selective β₂ sympathomimetic bronchodilator drugs.

Drugs of group 1.1 exert both α and β sympathomimetic effects. They include the drug substances adrenaline and ephedrine. Both adrenaline and ephedrine are known clinically as bronchodilators. Though adrenaline, despite side effect induced via its α-sympathomimetic properties, is still used by some practitioners for the treatment of acute asthma, both adrenaline and ephedrine have been largely superseded in asthma therapy.

The drugs of group 1.2 have both β₁ and β₂ sympathetic activity but no, or only limited, α-sympathomimetic activity. Of the group 1.2 drugs, isoprenaline is the best known representative. Isoprenaline differs from the drugs of group 1.3 in its faster onset but shorter duration of action and its cardiac stimulating effects which result largely from its β₁ activity. Though isoprenaline has previously been extensively used as bronchodilator therapy in asthma, its use has today become clinically restricted. Thus, in the UK, a rise in the rate of asthma death in the 1960's believed to have been specifically associated with isoprenaline usage has resulted in discontinuation of its clinical application.

The selective β₂ sympathomimetic bronchodilator drugs of group 1.3 (herein referred to for convenience collectively as “GROUP 1.3 DRUGS”) act, as their, name implies, selectively on the β₂ adrenergic receptors. The GROUP 1.3 DRUGS include for example, the drug substances a) TERBUTALINE, b) ALBUTEROL (also known as SALBUTAMOL), c) FENOTEROL, d) HEXOPRENALINE, e) RIMITEROL, f) ISOETHARINE, g) METAPROTERENOL, h) REPROTEROL, i) CLENBUTEROL, j) PROCATEROL, k) CARBUTEROL, l) TULOBUTEROL, m) PIRBUTEROL, n) BITOLTEROL and, more recently, the so-called “long acting selective β₂ sympathomimetic bronchodilator drug substances” o) FORMOTEROL, p) BAMBUTEROL and q) SALMETEROL [(R,S)-1-(4-hydroxy-3-hydroxymethylphenyl)-2-[6-(4-phenylbutoxy)hexylamino]ethanol]. All of the above recited GROUP 1.3 DRUGS are commercially available and clinically used, generally in pharmaceutically acceptable salt form, e.g. as the sulphate [(a), (b), (d) and (g)], hydrobromide [(c) and (e)], hydrochloride [(f), (h) to (l) and (p)], dihydrochloride [(d) and (m)], fumarate [(o)], methanesulfonate [(n)], hydroxynaphthoate [(q)] or, where appropriate, one or other of the hydrate forms thereof—see e.g. Merck Index, 11th edition (1989), items 9089 (a), 209 (b), 3927 (c), 4628 (d), 8223 (e), 5053 (f), 5836 (g), 8142 (h), 2347 (i), 7765 (j), 1840 (k), 9720 (l), 7461 (m), 1317 (n), 4159 (o) and 963 (p) and references cited therein and, for (q), Am. Rev. Resp. Dis. 137 (4; 2/2) 32 (1988).

Further GROUP 1.3 DRUGS currently in development include for example the drug substances r) BROXATEROL, s) ETANTEROL, t) IMOXITEROL, u) NAMINTEROL, v) PICUMETEROL, w) RP 58802 [Rhône-Poulenc], x) RU 42173-[Hoechst Roussel-Uclaf) and y) ZK 90055 [Schering].

GROUP 1.3 DRUGS characteristically contain as part of their structure an ethanolamine or 2-amino-ethanol moiety of formula I 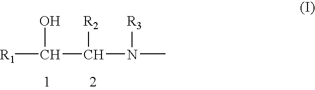
in which R₁ is an aromatic group.

Commonly R₁ is 3,4- or 3,5-dihydroxyphenyl as in the case of the GROUP 1.3 DRUGS (a), (c), (d), (e), (f), (g) and (h) above or 4-hydroxy-3-hydroxymethylphenyl as in the case of the GROUP 1.3 DRUGS (b) and (q). R₁ may also be, e.g., 2-hydroxymethyl-3-hydroxy-6-pyridyl; 3,4-ditoluoyloxy-phenyl; 3-formylamino-4-hydroxyphenyl; 3,5-N,N-dimethylcarbamoyloxyphenyl; 4-amino-3,5-dichlorophenyl; 4-hydroxy-3-ureidophenyl; or 2-chlorophenyl as in the case of the GROUP 1.3 DRUGS (l), (m), (o), (p), (i), (k) and (l) respectively.

R₃ in formula I is commonly H. An exception in this respect is the GROUP 1.3 DRUG (e) above. In this case R₂ and R₃ together are a group of formula —(CH₂)₄—.

R₂ in formula I is also commonly H. Exceptions in this respect are the GROUP 1.3 DRUG (e), as noted above, as well as (f) and (j) in both of which R₂ is ethyl.

Since the formula I moiety comprises at least 1 asymmetric carbon atom (C1 in formula I), all of the GROUP 1.3 DRUGS exist in optically active isomeric form, with the said carbon atom having the (R) or (S) configuration [as designated using the Cahn-Ingold-Prelog system (Angew. Chem. Intern. Ed. 5, 385-415 (1966)]. When the said carbon atom is the sole asymmetric carbon atom present, GROUP 1.3 DRUGS thus exist as individual (R) or (S) enantiomers or in racemic [(RS)] form, i.e. as a 50:50 mixture of the (R) and (S) enantiomers.

Individual GROUP 1.3 DRUGS in which R₂ in the formula I moiety is other than H or in which the remainder of the molecule includes an asymmetric carbon atom exist in a variety of isomeric forms, i.e. in individual (R,R), (S,S), (R,S) and (S,R) isomeric form, as racemic [(RS,RS) and (RS,SR)] mixtures comprising the (R,R) plus (S,S) and (R,S) plus (S,R) enantiomeric pairs, as well as in the form of diastereomeric mixtures comprising all four isomeric forms. This is so, for example, in the case of the GROUP 1.3 DRUGS (c), (d), (e), (f) and (o) above.

Individual enantiomers (e.g. (R) or (S), or (R,R) or (S,S) enantiomers] of GROUP 1.3 DRUGS are known and have been described together with processes for their production in the literature. Pharmacological studies and clinical, e.g. metabolic, investigations employing healthy volunteers have also been carried out using individual enantiomers of GROUP 1.3 DRUGS. It is furthermore known that the β₂ sympathomimetic/bronchodilator activity of GROUP 1.3 DRUGS resides primarily in individual enantiomers in which the hydroxy bearing carbon atom, C1 in formula I has the (R) configuration. The corresponding (S) enantiomer in contrast has no or very little bronchodilator activity. [See e.g. Murase et al., Chem. Pharm. Bull., 26 (4), 1123-112-9 (1-976); Hartley et al., J. Med. Chem. 14 (9), 895-896 (1-971); Okamoto et al., J. Liq. Chromatogr. 11, 2147-2163 (1988), Koster et al., Biochem Pharmacol., 35 (12), 1981-1985 (1986), Borgström et al., Br. J. Clin. Pharmac., 27, 49-56 (1989) and references therein.]

This knowledge notwithstanding, GROUP 1.3 DRUGS are marketed and employed for regular clinical usage, e.g. in the treatment of obstructive or inflammatory airways disease, in racemic [(RS)] form, that is as mixtures of the bronchodilatorily active (R) and inactive (S) enantiomeric pairs. [In the case of GROUP 1.3 DRUGS comprising two asymmetric carbon atoms the clinically employed racemic mixture is commonly that comprising the (R,R) plus (S,S) enantiomeric pair, i.e. the (RS,RS), racemate, as in the case of the so called “A racemate” of FENOTEROL—cf. Merck. Index, Loc. cit.]

The GROUP 1.3 DRUGS can be administered orally, parenterally or (most commonly) by inhalation, e.g. using nebulisers or metered aerosol devices or as inhaled powders. Inhalation of GROUP 1.3 DRUGS presently represents the mainstay of bronchodilator therapy for the treatment of asthma of all grades of severity. The duration of bronchodilatation induced by the majority of GROUP 1.3 DRUGS is relatively short and they are employed to relieve asthma attack as and when it occurs. As indicated above, the more recently introduced GROUP 1.3 DRUGS, e.g. (o), (p) and (q) above, are characterised by their longer duration of action and hence apparent reduced frequency of dosaging required.

Although the GROUP 1.3 DRUGS are effective and generally seem to be well tolerated, their safety, especially at high dosages, has been questioned over many years and numerous reports have appeared on the adverse effects of GROUP 1.3 DRUG therapy (see e.g. Paterson et, al: “American Review of Respiratory Disease, 120, 84.4 to 1187 (1979) especially at p.p. 1165 et seq.). More recently, from New Zealand, where a continuing increase in asthma death has been recorded, two case control studies reported in the Lancet have linked increase in asthma mortality to use of the GROUP 1.3 DRUG, FENOTEROL—see in particular: Editorial “β₂ agonists in asthma: relief, prevention, morbidity”, Lancet, 336, 1411-1412 (1990). A subsequently reported Canadian study finds that the use of inhaled GROUP 1.3 DRUGS, principally FENOTEROL and ALBUTEROL, is associated with “an increased risk of the combined outcome of fatal and near-fatal asthma, as well as of death from asthma alone”—see Spitzer et al., New England J. of Med., 326 (8), 501-506 (1992) and the Editorial to the same issue at page 560.

Various possible explanations for observed episodes of increased airway obstruction, arterial hypoxaemia or “anomolous” or “paradoxical” bronchospasm, as well as increased morbidity associated with GROUP 1.3 DRUG usage, in particular long term/high dose usage, have been proposed.

These have included, for example, reactive myogenic tone, increased inflammatory burden, adrenoceptor tachyphylaxis and induction of airway hyperreactivity, as well as the involvement of spasmogenic drug metabolic products or long term influence of aerosol spray propellants—see e.g. Paterson et al. loc. cit. and Morley et al. Eur. Respir. J., 3, 1-5 (1990).

As already noted, an increase in asthma death had earlier been associated with use of the GROUP 1.2 DRUG isoprenaline. Isoprenaline is metabolised in part by the enzyme catechol-O-methyl transferase, giving a 3-methoxy derivative which has β-adrenoceptor antagonist activity. It has, for example been suggested that it is this metabolite which was the cause of difficulty. More recently it has been proposed that isoprenaline-induced asthmatic exacerbation is due to an exacerbation of airways-hyperreactivity or inflammatory status common to the (S) [or (+)] and (R) [or (−)] enantiomers of isoprenaline (see e.g.: Mazzoni et al., Brit. J. Pharmacol, 91, 326 (1987); Morley et al., J. Physiol; 390, 180 P (1987) and Lancet, Jul. 16, 1988, p. 160; and Sanjar et al., J. Physiol, 425, 43-54 (1990)-isoprenaline like the GROUP 1.3 DRUGS was employed clinically in (RS) racemic [or (+)] form.] No consensus on the subject has however been reached within the scientific community and no evidence has hitherto been adduced which might link experience with isoprenaline to that with GROUP 1.3 DRUGS.

At the same time there is mounting concern within the medical profession as to the potential dangers of GROUP 1.3 DRUG usage in asthma therapy. To quote the Lancet Editorial already referred to:

“These studies raise serious question about the use of β₂ agonists [i.e. GROUP 1.3 DRUGS]. The findings of Sears et al. could be interpreted as supporting the current trend towards earlier use of corticosteroids and other preventers of inflammation [for asthma therapy] rather than perseverance with an escalating bronchodilator regimen. The findings of the Nottingham and Dunedin groups also indicate that there is some way to go before long acting β₂ agonist preparations such as salmeterol and formoterol can be unreservedly recommended for routine use in the management of asthma. There seem to be clear advantages of compliance and possibly of anti-inflammatory activity associated with such agents, but the potential for adverse effects cannot be ignored. Clinicians researchers and pharmaceutical companies must now attempt to redefine the use of β₂ agonists in asthma.” [Emphasis added.]

Equally there has been evident inability or reluctance to conceive of any problem in relation to GROUP 1.3 DRUG therapy as being inherent in GROUP 1.3 DRUGS themselves or as hitherto employed—cf. the following, taken from the Editorial to the New England Journal of Medicine also previously referred to: “Although . . . too much reliance is placed on beta-agonists [GROUP 1.3 DRUGS], it is difficult to believe that the problem is related directly to the more regular use of inhaled beta-agonists.”

In accordance with the present invention it has now been found that, whereas bronchodilator efficacy of GROUP 1.3 DRUGS is associated with, or associated primarily with, one optically active enantiomer, the bronchodilatatory less active or inactive enantiomer or antipode induces an adverse effect, e.g. in asthma. (This finding does not, of course, exclude the possibility that the isomer having bronchodilator efficacy may also possess adverse pharmacological properties which are masked or compensated for by its beneficial bronchodilator efficacy.) The present invention thus surprisingly teaches that the long-standing problems inherent in GROUP 1.3 DRUG therapy may unexpectedly be met or ameliorated by the relatively simple expedient of administering GROUP 1.3 DRUGS not, as hitherto, in the form of a racemic mixture but in the form of the individual bronchodilatatory effective enantiomer (referred to hereinafter for convenience as the “BRONCHODILATOR ENANTIOMER”).

While the suitability, in particular of high-dose or long-term, GROUP 1.3 DRUG therapy has long been a subject of debate and, more recently, acute question, the practice of administering drugs of this group as racemic mixtures has continued. This practice has been accepted by drug registration authorities world-wide and even the most recently introduced of the GROUP 1.3 DRUGS have been developed for clinical use as racemic mixtures.

This practice is based upon the assumption or understanding that the non-bronchodilator component of the racemic mixture, i.e. the bronchodilatorily less or inactive enantiomer or antipode of the BRONCHODILATOR ENANTIOMER is devoid of any relevant drug effect and can thus be administered together with the BRONCHODILATOR ENANTIOMER essentially as inactive ballast and without risk to the patient. The teaching of the invention thus stands in stark opposition to long, widely established and continuing practice.

While simple in conception, the present invention thus runs contrary to to the wisdom of the art. In that the GROUP 1.3 DRUGS clearly offer very considerable potential benefit for bronchodilator usage in asthma, the need to find a means of avoiding, ameliorating or restricting disadvantages inherent in their use is urgent and crucial. By meeting this need, the present invention may be anticipated to bring immeasurable benefit both to the medical profession and the world asthma population.

In accordance with the foregoing the present invention provides:
A An improved (e.g. safer) method of treating inflammatory or obstructive airways disease or a method of treating inflammatory or obstructive airways disease with the avoidance, amelioration or restriction of deleterious side effect, in a human subject in need thereof, which method comprises administering to said subject a GROUP 1.3 DRUG, said GROUP 1.3 DRUG being administered predominantly in the form of its BRONCHODILATOR ENANTIOMER; or, in the alternative:
B A GROUP 1.3 DRUG predominantly in the form of its BRONCHODILATOR ENANTIOMER for use in the improved (e.g. safer) treatment of inflammatory or obstructive airways disease in humans, or for use in the treatment of inflammatory or obstructive airways disease in humans to avoid, ameliorate or restrict deleterious side effect, or for use in the preparation of a pharmaceutical composition for use in such treatment.

GROUP 1.3 DRUGS to which the present invention applies include any selective β₂ sympathomimetic bronchodilator drug comprising an ethanolamine moiety, e.g. of formula I as illustrated above wherein R₁ is an aromatic group, for example a moiety of formula I as illustrated above wherein R₁, R₂ and R₃, individually or collectively have any one or more of the meanings hereinbefore recited.

Specific GROUP 1.3 DRUGS to which the present invention applies include any of the drug products (a) through (y), especially (a) through (q) hereinbefore-identified and, in particular, (b) ALBUTEROL and the “long acting” GROUP 1.3 DRUGS, in particular (o) FORMOTEROL, (p)-BAMBUTEROL and (q) SALMETEROL. The invention is to be understood as relating to GROUP 1.3 DRUGS both in free form as well as pharmaceutically acceptable acid addition salt form, e.g. as hereinbefore set forth for the GROUP 1.3 DRUGS (a) through (q), and including hydrate forms thereof. All references to GROUP 1.3 DRUGS, whether individually or collectively and in whatever manner, in relation to the present invention both herein and in the accompanying claims are to be understood accordingly as embracing such salt and hydrate forms.

As hereinbefore described in relation to formula I, C1 in BRONCHODILATOR ENANTIOMER of GROUP 1.3 DRUGS characteristically has the (R) configuration. In the case of GROUP 1.3 DRUGS having a single asymmetric carbon atom BRONCHODILATOR ENANTIOMER will thus be the (R) enantiomer. In the case of GROUP 1.3 DRUGS having two asymmetric carbon atoms BRONCHODILATOR ENANTIOMER will be the (R,R) or (R,S) isomer. In practice, GROUP 1.3 DRUGS having two asymmetric carbon atoms have hitherto been used in clinic generally in the form of the (RS,RS) racemic mixture and it is the (R,R) enantiomer which generally has the greatest bronchodilator potency (see e.g. Murase et al., loc. cit.). In the case of GROUP 1.3 DRUGS-having two asymmetric carbon atoms BRONCHODILATOR ENANTIOMER will thus usually be the (R,R) enantiomer.

In practicing the present invention, GROUP 1.3 DRUG is employed predominantly in the form of its BRONCHODILATOR ENANTIOMER. Preferably GROUP 1.3 DRUG will be employed in the form of its pure or substantially pure BRONCHODILATOR ENANTIOMER, that is in a form free or substantially free of other isomeric forms, in particular of the chirally opposite (“non-bronchodilator”) antipode. Suitably GROUP 1.3 DRUGS will comprise at least >75%, preferably at least 90%, e.g. >95% or >98% BRONCHODILATOR ENANTIOMER. As previously indicated GROUP 1.3 DRUGS in pure or substantially pure isomeric form are known [see for example Murase et al. and Hartley et al. loc. cit. and other references referred to in the Merck Index hereinbefore cited] or may be obtained analogously, e.g. by resolution of diastereomeric salt forms/chromatographic techniques.

The present invention provides a method or use for the treatment of inflammatory airways disease, in particular for effecting bronchodilatation, e.g. as a means of alleviating airways obstruction, in particular acute airways obstruction, e.g. asthma attack, occurring in such disease.

The invention thus provides symptomatic, rather than prophylactic, therapy for such disease.

The teaching of the present invention is applicable in the therapy of inflammatory obstructive airways disease, in particular any such disease for which GROUP 1.3 DRUG therapy is commonly practiced, for example chronic obstructive pulmonary disease, e.g. consequential to cystic fibrosis, emphysema and, especially, chronic bronchitis and, most especially, asthma.

The present invention avoids deleterious side effects hereinbefore resulting or observed in, e.g. asthmatic, patients consequent to conventional clinical usage of GROUP 1.3 DRUGS as racemic mixtures. In particular the invention provides means to avoid, ameliorate or restrict deleterious side effect, e.g. side effect deleterious to the airways. Thus the invention provides means to avoid, ameliorate or restrict exacerbation of disease status, for example basal disease, e.g. basal asthmatic, status or to avoid, ameliorate or restrict compromise or deterioration of lung function, or any other side effect concomitant to conventional clinical usage, for example “anomolous”, “rebound” or “paradoxical” bronchospasm and, especially, increase in airway obstruction, exacerbation of late asthmatic response or non-specific bronchial reactivity or arterial hypoxaemia. Without limiting the present invention to any specific theory or mode of action, the present invention is in particular to be understood as providing a means for the avoidance, amelioration or restriction of exacerbation of airways hyperreactivity and/or of inflammatory or other event associated with, or which is an aetiological component of inflammatory or obstructive airways disease, e.g. asthma. Such events are to be understood as including for example, inflammatory cell infiltration of the lungs or airways, connective tissue deposition or smooth muscle hyperplasia within the lungs or airways or other morphological change associated with asthmatic status. The present invention also provides a means of preventing or reducing-morbidity, e.g. asthma morbidity, ascribable to conventional, e.g. high dosage or long term, GROUP 1.3 DRUG usage.

The present invention is especially applicable in the therapy of bronchial asthma of whatever type or genesis. It is applicable to both intrinsic and extrinsic asthma. It is especially applicable to the treatment of allergic or atopic (i.e. IgE-mediated) asthma or non-atopic-asthma, as well as exercise-induced asthma, occupational asthma, asthma induced following bacterial infection or drug, e.g. aspirin, ingestion and other non-allergic asthmas. Treatment of asthma is also to be understood as embracing treatment of subject's, e.g. of less than 4 or 5 years of age, exhibiting chronic cough or wheezing symptoms, in particular at night, and diagnosed or diagnosable as “wheezy infants”, i.e. as embracing the treatment of “wheezy infant syndrome”. Other diseases to which the present invention is in particular applicable include for example chronic obstructive pulmonary or airways disease (COPD or COAD).

As previously mentioned, the present invention embraces the understanding that BRONCHODILATOR ENANTIOMERS of GROUP 1.3 DRUGS may themselves-exhibit adverse pharmacological properties in common with the non-bronchodilator antipodes, which are masked, or compensated for, by their bronchodilator efficacy. As a direct corollary to this and in the light of the understanding of said adverse effects as taught by the invention, the therapeutic benefit of BRONCHODILATOR ENANTIOMERS may be yet further improved by co-administration of drug substances capable of reversing or inhibiting the development of airways hyperreactivity, notably the drug substance KETOTIFEN (cf. Merck index, loc. cit. item 5187). Accordingly in a further aspect the present invention provides:
C A method as defined under A above, which method additionally comprises administration of KETOTIFEN; or
D A GROUP 1.3 DRUG predominantly in the form of its BRONCHODILATOR ENANTIOMER for use as defined under B above, wherein said use comprises use in conjunction with use of KETOTIFEN, i.e. additionally comprises administration of KETOTIFEN.

KETOTIFEN is known and commercially available, e.g. in pharmaceutically acceptable acid addition salt form, for example as its hydrogen fumarate, for use, inter alia, as an asthma prophylactic drug. References to KETOTIFEN herein are to be understood as embracing KETOTIFEN in free base form or in the form of any of its pharmaceutically acceptable acid addition salts.

For the above purposes KETOTIFEN will generally be administered in anti-asthmatically effective amount, i.e. at dosages conventionally administered for the prophylaxis of asthma, as hereinafter described. In practicing the invention KETOTIFEN may be administered either concomitantly with or independently of BRONCHODILATOR ENANTIOMER of GROUP 1.3 DRUG, e.g. in a separate daily regimen during the course of therapy employing BRONCHODILATOR ENANTIOMER of GROUP 1.3 DRUG.

The deleterious effects of the non-bronhcodilator enantiomer (i.e. antipode of BRONCHODILATOR ENANTIOMER) of GROUP 1.3 DRUGS, e.g. of (S)-ALBUTEROL and (S)-TERBUTALINE [the dextro or (+) optically active isomers] as well as the advantages obtaining from the application of the present invention may be demonstrated in conventional animal models as well as in clinical trials for example as follows:

### EXAMPLE 1

### Influence of Non-Bronchodilator Enantiomers of GROUP 1.3 DRUGS on Airways Hyperreactivity in the Guinea Pig

Guinea-pigs (circa 500 g) are anaesthetised by intraperitoneal injection of sodium phenobarbitone (100 mg/kg) and sodium pentobarbitone (30 mg/kg) then paralysed by intramuscular injection of gallamine (10mg/kg). Animals are ventilated (8 ml/kg, 1 Hz) via a tracheal cannula using a mixture of air and oxygen (50:50, v/v). Ventilation is monitored at the trachea by a pneumotachograph (type 0000, Fleisch, Zabona A. G., CH) connected to a differential pressure transducer (type MP 4514871, Validyne, USA). Coincident pressure changes within the thorax are measured via an intrathoracic cannula, using a differential pressure transducer (type MP 4524, Validyne, USA); blood pressure and heart rate are recorded from the carotid artery using a pressure transducer (type P23Dd, Gould, USA). From measurements of air-flow and intrathoracic pressure, both airway resistance (R_{L}) and compliance (C_{d y n}) are calculated at each respiratory cycle using a digital electronic pulmonary monitoring system (PMS, Mumed Ltd, London, UK) and recorded. Blood pressure, intrathoracic pressure, airflow and computed R_{L} and C_{d y n} in real time are displayed on a visual display unit (model AT3, IBM, USA). Experimental data is stored electronically and experimental traces or processed data are plotted on a laser printer (Laser Jet Series II, Hewlett Packard, USA) as required.
1) In a first series of experiments responsivity of the airways to intravenous injection of histamine (0.56-1.8 μg/kg at 10 min. intervals) is defined before, and twenty minutes after, intravenous infusion of (S)-ALBUTEROL over one hour (total dose 100 μg/kg). Increase of airway resistance following intravenous injection of histamine (0.56, 1.0 & 1.8 μg/kg) in one experimental run is recorded as (10±1.8, 41.03±9.14 & 223±69.91 cmH₂ 0/1/sec.) before and (60.01±12.86, 149.06±31.64 & 539±185.14 cmH₂ 0/1/sec.) after infusion of (S)-ALBUTEROL (10.0 μg/kg). Incremental differences for successive doses of histamine recorded are 50.1, 108.03 & 316 cmH₂ 0/1/sec. By comparison, increased airway resistance in response to intravenous injection of histamine (0.56, 1.0 & 1.8 μg/kg) before and after intravenous infusion of vehicle (0.9% saline) is recorded as (7.05±1.7, 21.68±3.05, 86.45±14.13 and 15.04±2.57, 30.42±5.39, 101±20 respectively) so that incremental differences for successive doses of histamine are 7.99, 8.74 & 14.75 cmH₂ 0/1/sec.
2) In a second series of experiments employing guinea pigs actively sensitised to ovalbumin [as described in Sanjar et al., Br. J. Pharmacol. 99, 679-68.6 (1990)], responsivity of the airways to intravenous injection of histamine (as under 1 above) before and after intratracheal (as under 1 above) before and after containing (S)-ALBUTEROL (10 μg) or (S)-TERBUTALINE (10 μg) is defined. In this test model both (S)-ALBUTEROL and (S)-TERBUTALINE are found to induce significant increase of airway resistance on intravenous injection of histamine as compared with animals receiving tragant only.

Similar or equivalent results are obtained employing non-bronchodilator enantiomer of other GROUP 1.3 DRUGS, e.g. the (S) or (S,S) enantiomer of GROUP 1.3 DRUGS (c) to (q) as hereinbefore set forth, at the same or equivalent dosage rates.

### EXAMPLE 2

### Influence of Non-Bronchodilator Enantiomer of GROUP 1.3 DRUGS on the Lung Function of Asthmatic Patients

The trial is carried out in double blind, placebo controlled format. Subjects are stable asthmatics with evident on-going compromisation of lung function. Typical subjects include allergic asthmatics or non-allergic (intrinsic asthmatics) with no evidence of atopy, clinically stable and using conventional nebulised GROUP 1.3 DRUGS therapy-regularly. Asthma medication is withdrawn ca. 12 hours prior to investigation and pulmonary function (FEV₁) is monitored at regular intervals prior to and following adminsitration of test substance or placebo (vehicle). Additionally PD20 for histamine is determined by measuring the effect of inhaled aerosols of histamine solutions (0.0625-8 mg/ml) 0.5 hrs before as well as 2.5 and 7.5 hrs after exposure to test substance/vehicle.

Test substance comprises GROUP 1.3 DRUG administered by the inhaled route either in racemic form (in accordance with conventional practice) at conventional single dose level or in substantially pure non-bronchodilator enantiomeric form at 0.25 to 0.5× the conventional single dose level.

In subjects receiving GROUP 1.3 DRUG in conventional, racemic form, e.g. receiving (R,S)-ALBUTEROL, (R,S)-TERBUTALINE or (RS,RS)-FENOTEROL, dose related reduction of airflow obstruction is observed as compared with subjects receiving placebo. Results thus accord with conventional observations for GROUP 1.3 DRUG therapy.

In subjects receiving GROUP 1.3 DRUG in substantially pure non-bronchodilator enantiomeric form, e.g. receiving (S)-ALBUTEROL, (S)-TERBUTALINE or (S,S)-FENOTEROL, after potential transient reduction in airflow obstruction attributable to any BRONCHODILATOR ENANTIOMER present in the Administered material, individual subjects exhibit a sustained fall in FEV₁, accompanied by increased wheezing and discomfort as compared with results obtained from subjects receiving placebo.

In practicing the present invention, BRONCHODILATOR ENANTIOMER of GROUP 1.3 DRUG may be administered in any form or by any route known or conventionally employed in relation to use of selected GROUP 1.3 DRUG in conventional racemic form, e.g. orally in the form of tablets, capsules, syrups, granulates and micro-granulates etc., intravenously in the form of an injectable solution, or by the pulmonary route. Preferably BRONCHODILATOR ENANTIOMER of GROUP 1.3 DRUG will be administered via the pulmonary route, e.g. by inhalation from an appropriate dispenser device, e.g., as hereinbefore indicated or as otherwise known or used in the art.

Dosages of BRONCHODILATOR ENANTIOMER of GROUP 1.3 DRUG employed in practicing the present invention will vary, e.g. depending on the particular GROUP 1.3 DRUG selected, the selected route of administration, the particular condition to be treated, the severity of the condition to be treated and the effect desired. In general however dosages of BRONCHODILATOR ENANTIOMER of the selected GROUP 1.3 DRUG will be of the order of about 40% to 60%, e.g. about 50%, of dosages-administered employing the same GROUP 1.3 DRUG in conventional, racemic form. This lowering of the dosage may readily be achieved, e.g. by preparing galenic forms comprising BRONCHODILATOR ENANTIOMER of the selected GROUP 1.3 DRUG as active ingredient in the same concentration as in conventionally employed dosage forms and reducing the daily dosaging requirement by ca. 50%, or by preparing galenic forms comprising BRONCHODILATOR ENANTIOMER as active ingredient at ca. 50% of the concentration conventionally employed for GROUP 1.3 DRUG and maintaining conventional daily dosaging requirements. In the latter case, the 50% reduction in active ingredient content will be compensated by the addition of the equivalent amount of an appropriate, inert pharmaceutically acceptable diluent or carrier.

Thus for administration by inhalation, (R,S)-ALBUTEROL is conventionally administered, e.g. via a metered dose aerosol delivering 100 μg racemic drug substance per actuation. For adults, administration is conventionally effected 3 to 4 times/day with 2 actuations at each administration, to give a dosage per-administration of 200 μg drug substance. The canisters employed in the delivery device contain ca. 20 mg (R,S)-ALBUTEROL or sufficient for 200 actuations.

Employing pure or substantially pure (R)-ALBUTEROL in accordance with the present invention, administration can be effected employing, an identical regimen to that used for the racemate but using canisters containing ca. 10 mg (R)-ALBUTEROL, giving a metered dose of 50 μg drug substance per actuation or a dosage of 100 μg drug substance 3 to 4 times/day, or using canisters containing ca. 20 mg (R)-ALBUTEROL, giving a metered dose of 100 μg drug substance per actuation and applying only 1 instead of 2 actuations at each administration.

From the foregoing it will be appreciated that suitable galenic formulations for practicing the present invention may be in all material respects identical to those employed for delivery of conventional, racemic GROUP 1.3 DRUG, but with appropriate compensation for reduction in active ingredient content where required.

As previously indicated, in practicing the present invention, BRONCHODILATOR ENANTIOMER of GROUP 1.3 DRUG is preferably administered by the pulmonary route, e.g. by inhalation. Compositions employed will thus preferably be in a form permitting, enabling or adapted for administration via the pulmonary route. Such forms will in particular include free flowing, or freely flowable, dispersible forms, for example liquid or finely divided powder forms, capable of or adapted to delivery as an inhalable spray, mist or dispersion in air, e.g. following delivery from an appropriate, e.g. aerosol, atomiser, dry powder dispenser or like device. Carriers, excipients, diluents etc. employed in such compositions will likewise preferably be selected from amongst those known, employed and/or recognised as suitable for pulmonary administration.

The following examples are illustrative of compositions suitable for use in accordance with the present invention:

### EXAMPLE 3

3.1 Tablets or capsules may contain the active agent in admixture with conventional pharmaceutically acceptable excipients, e.g. inert-diluents such as calcium carbonate, sodium carbonate, lactose and talc, granulating and disintegrating agents, e.g. starch and alginic acid; flavouring, colouring and sweetening agents, binding agents, e.g. starch, gelatin and acacia, and lubricating agents, e.g. magnesium stearate, stearic acid and talc, e.g. as follows:

| INGREDIENTS | WT./DOSE | |
|---|---|---|
| (R)-METAPROTERENOL (as its sulfate) | 20.00 | mg |
| in substantially pure form | | |
| Lactose (200 mesh) | 90.00 | mg |
| Corn starch | 35.00 | mg |
| Silicon dioxide (Aerosil 200) | 1.75 | mg |
| Magnesium stearate | 3.25 | mg |
| TOTAL | 150.00 | mg |

The ingredients are intimately admixed employing conventional galenic procedures, filled into hard gelatin capsules and the capsules sealed.

The capsules are useful in accordance with the present invention in the therapy of asthma on administration in adults 2× daily to give a daily dose of 40 mg/day/p.o. Alternatively capsules may be prepared comprising 10.00 mg (R)-ORCIPRENALINE (as its sulfate) for administration in adults 4× daily.

Equivalent oral compositions may be prepared comprising BRONCHODILATOR-ENANTIOMER of any other GROUP 1.3 DRUG, e.g. as hereinbefore referred to, either at conventional unit dosage drug concentration* for administration at 50% conventional dosaging rate* or at 50% conventional unit dosage drug concentration for administration at conventional dosaging rate.
[*For the drug substances TERBUTALINE, FENOTEROL and CARBUTEROL fore example, conventional oral unit dosage forms (comprising racemic material) comprise 2.5 or 5.0 mg; 5.0 or 10.0 mg; and 2.3 mg racemic material respectively, for administration 2 to 4× daily.]

3.2 Inhalable aqueous solutions may also be prepared in conventional manner, e.g. optionally with the addition of ethanol as solubilizer, and with acid buffering agents to an end pH of 4.0. Stabilizing and preserving agents may also optionally be added. Suitable compositions for pulmonary application from a conventional metered delivery device may be made up for example as follows:

Aqueous solutions are prepared comprising (a) 0.5, (b) 1.0 or (c) 2.0 mg (R)-ALBUTEROL as the sulphate/ml and adjusted to pH ca. 4.0 by the addition of H₂SO₄. Compositions are filled in 2.5 ml amounts, comprising 0.5%, 1.0% and 2.0% (R)-ALBUTEROL, into plastic-ampoules for insertion into a conventional metered device, e.g. for use, in relation to composition (a) with 2× actuation delivering a total of 100 μg (R)-ALBUTEROL 2 to 4× daily, in relation to composition (b) with 1× actuation delivering a total of 100 μg (R)-ALBUTEROL 2 to 4× daily or in relation to composition (c) with 1× actuation delivering a total of 200 μg (R)-ALBUTEROL 1 to 2× daily.

Equivalent compositions may be prepared comprising BRONCHODILATOR ENANTIOMER of any other GROUP 1.3 DRUG, e.g. as hereinbefore referred to, either at conventional unit drug concentration** for administration at 50% conventional dosaging rate or at 50% conventional drug concentration for administration at conventional dosaging rate.
[**For the drug substances ISOETHARINE, METAPROTERENOL, TERBUTALINE, FENOTEROL and CARBUTEROL for example, conventional inhaled doses (per puff) are 350 μg; 650 μg; 250 μg; 200 μg; and 100 μg racemate respectively, for use in two puffs generally administered 2 to 4 or up to 6× daily.]

In accordance with the foregoing the present invention also provides:
E A pharmaceutical composition comprising a GROUP 1.3 DRUG predominantly in the form of its BRONCHODILATOR ENANTIOMER as active ingredient, together with a pharmaceutically acceptable diluent or carrier therefor.

Pharmaceutical compositions are to be understood as being, in particular, compositions of which the individual components are not only suitable or allowable for therapeutic usage but which are manufactured and processed under conditions of sterility appropriate or required for therapeutic usage.

When the method of the present invention is practiced in conjunction KETOTIFEN therapy, dosages of KETOTIFEN employed will generally be the same or of similar order to KETOTIFEN dosages as conventionally employed for the prophylaxis or management of asthma, that is of the order of 1 to 4 mg, preferably 2 or 4 mg/day/p.o., suitably administered in 1 or 2 mg doses, preferably 1× or 2× daily, or in liquid. e.g. syrup form. Suitable oral dosage forms, e.g. 1 mg and 2 mg tablets and capsules as well as syrup formulations comprising KETOTIFEN as active ingredient, for use in practicing the present invention are known and commercially available.

Utility of the present invention may also be demonstrated in clinical trials, for example, performed as follows:

Clinical Trial I

Trial subjects are selected from patients having a clinical history of asthma and demonstrable airway obstruction (e.g. FEV₁ less, than predicted from standard tables) that is resolved by inhalation of clinical doses of GROUP 1.3 DRUGS in conventional, racemic form [e.g. of (R,S)-ALBUTEROL]. Subjects also exhibit demonstrable increase in airway reactivity to inhaled histamine or methacholine. Typically, selected subjects are young adults (ca. 15 to 25 years of age) allergic to pollens, animal danders or house dust mite, using inhaled conventional, racemic GROUP 1.3 DRUG therapy intermittently (e.g. according to subjective perception of symptoms), with or without additional anti-asthma therapy such as inhaled steroid, cromoglycate or KETOTIFEN.

Trial subjects are divided into separate groups receiving either conventionally, racemic GROUP 1.3 DRUG [e.g. (R,S)-ALBUTEROL] at conventional doses of 200 μg or BRONCHODILATOR ENANTIOMER of GROUP 1.3 DRUG dosing [e.g. (R)-ALBUTEROL] at 50% doses of 100 μg, all doses administered by inhalation regularly, e.g. 2 to 4× daily over a period of 1 to 6 months. Concomitant additional therapy, as mentioned above is maintained where used. Subjects are monitored at monthly intervals during the course of the trial period for airways hyperreactivity, preferably using leukotriene C₄ or E₄ as test spasmogen, e.g. as reported in references already referred to hereinbefore.

Increase in airway hyperreactivity is evidenced in subjects receiving conventional, racemic GROUP 1.3 DRUG. Subjects receiving BRONCHODILATOR ENANTIOMER in contrast exhibit a clearly restricted tendency to increase in hyperreactivity but exhibit, equivalent benefit in terms of bronchodilator action during exacerbation. In subjects receiving concomitant KETOTIFEN yet further restricted trend towards increase in hyperreactivity is observed.

Clinical Trial II

Subjects are selected from patient groups as described for TRIAL I. Subjects receive conventional, racemic GROUP 1.3 DRUG [e.g. (R,S) ALBUTEROL at 200 μg by inhalation] or BRONCHODILATOR ENANTIOMER of GROUP 1.3 DRUG [e.g. (R)-ALBUTEROL at 100 μg by inhalation]. The alternative therapies are assigned to individual subjects in randomized, double-blind manner. Pulmonary function (e.g. FEV₁) and sensitivity to a test of airway hyperreactivity (e.g. inhaled aerosolised histamine) is determined before drug-administration and after intervals (e.g. of 2 and 5 hours) post drug-administration.

In the case of subjects receiving conventional, racemic GROUP, 1.3 DRUG, evident mismatch is recorded between observed drug bronchodilator efficacy and suppression of manifestation of hyperreactivity, such that there is no observed protection from manifestation of hyperreactivity even though substantial bronchodilator response remains evident. In subjects receiving BRONCHODILATOR ENANTIOMER, degree of mismatch is significantly reduced while bronchodilator efficacy is maintained.

1-10. (canceled)

11. A method of treating inflammatory or obstructive airways disease while avoiding, ameliorating or restricting side effects associated with the administration of racemic terbutaline, the method comprising administering to a human subject in need of such treatment, a therapeutically effective amount of terbutaline, the amount comprising greater than 98% R-terbutaline.

12. The method according to claim 11, wherein the side effects avoided, ameliorated or restricted are deleterious to the airways.

13. The method according to claim 11, wherein the side effect is exacerbation of basal disease status or compromise or deterioration of lung function.

14. The method according to claim 11, further comprising the administration of an anti-asthmatic amount of ketotifen.

15. An inhalable terbutaline composition comprising greater than 98% R-terbutaline and a pharmaceutically acceptable carrier.
